(19) United States
(12) Patent Application Publication
(10) Pub. No.: US 2026/0124261 A1
(43) Pub. Date: May 7, 2026

(54) STRAIN OF SACCHAROMYCES CEREVISIAE VAR. BOULARDII FOR TREATING INFECTIOUS DISEASES OF THE ORAL CAVITY

(71) Applicant: LESAFFRE ET COMPAGNIE, PARIS (FR)

(72) Inventors: Nathalie BALLET, MARCQ EN BAROEUL (FR); Amélie DECHERF, BONDUES (FR); Elodie DEHAY-HARMEL, LOMPRET (FR)

(73) Assignee: LESAFFRE ET COMPAGNIE, PARIS (FR)

(21) Appl. No.: 19/439,566

(22) Filed: Jan. 5, 2026

Related U.S. Application Data
(62) Division of application No. 17/414,084.

(30) Foreign Application Priority Data
Dec. 19, 2018 (FR) 18 73321

(51) Int. Cl. A61K 36/064; A61K 9/00; A61K 45/06; A61P 31/04

(52) U.S. Cl. CPC A61K 36/064; A61K 9/006; A61K 45/06; A61P 31/04

(57) ABSTRACT

A yeast strain of Saccharomyces cerevisiae var. Boulardii for the treatment and/or the prevention of infectious diseases of the oral cavity, such as dental caries and periodontal diseases. This strain can be used either alone or in combination with the inactive dry form of a yeast strain of Saccharomyces cerevisiae.

### RELATED PATENT APPLICATIONS

The present patent application is a divisional of U.S. patent application Ser. No. 17/414,084, filed on Jun. 15, 2021, which is filed pursuant to 35 U.S.C. § 371 as a U.S. National Phase application of International Patent Application No. PCT/EP2019/086265, which was filed on Dec. 19, 2019, claiming the benefit of priority to French patent application number FR 18 73321 filed on Dec. 19, 2018. The content of each of the aforementioned patent applications is incorporated herein by reference in its entirety.

### FIELD

The present invention relates to the field of oral health. More particularly, the invention relates to a Saccharomyces cerevisiae var. boulardii strain, used alone or in combination with an inactivated dry Saccharomyces cerevisiae yeast, in the treatment and/or prevention of infectious diseases of the oral cavity, such as dental caries and periodontal disease.

### BACKGROUND

In man, the oral cavity is an ecosystem which is complex, open to the interior, and inhabited by numerous microorganisms including more than 700 bacterial species detected (Ghannoum et al., PLOS Pathog., 2010; 6: e1000713; Marsh et al., Periodontol., 2000, 55:16-35; Paster et al., Periodontol., 2000, 42:80-87). For a given individual, the number of resident bacterial species varies from 150 to 250. Most of these species are commensal and necessary to maintain the balance of this ecosystem. However, in certain situations (high carbohydrate intake, tobacco use, or physiological changes such as aging, puberty or pregnancy, etc.), a disruption of this balance occurs and can lead to the development of infectious diseases of the oral cavity.

The main infectious diseases of the oral cavity are dental caries and periodontal disease. Dental caries are a polybacterial disease that manifests itself by a demineralization of the hard tissues of the tooth. This demineralization is due to the production of acids by the fermentative bacteria present in the dental plaque. Mutans group streptococci and lactobacilli are considered as the main cariogenic bacteria. The World Health Organization (WHO) report on oral health notes the high prevalence of caries, in particular among the youngest. Compared with the rest of the world, the risk of childhood caries is much higher in developed countries (North America, Australia, Europe, Japan), due to a diet very high in sugar in these countries.

Periodontal diseases are a group of pathologies affecting the periodontium (i.e., the tissues supporting the tooth-bone and gingival mucosa). These diseases are divided into two main categories: gingivitis and periodontitis. Gingivitis is any inflammation limited to the superficial periodontium. Periodontitis is an advanced infectious lesion of the periodontium and often follows gingivitis. The presence of certain bacteria and an intense inflammatory response lead to the destruction of the periodontium. The change from a healthy state to a state of periodontal disease is accompanied by a progressive transition to a flora richer in anaerobic and Gram-negative bacteria. This phenomenon is called anaerobic drift. The bacterial species most frequently involved in periodontal disease include: Fusobacterium nucleatum and Porphyromonas gingivalis whose co-aggregation seems to improve their survival and pathogenicity (Diaz et al., Microbiology, 2002, 148:467-472; Saito et al., FEMS Immunol. Med. Microbiol., 2008, 54:349-355; Polak et al., J. Clin. Periodontol., 2009, 36:406-410).

The treatment of dental caries, which affect only the enamel or dentin, includes a filling with a composite or silver amalgam. When the tooth is severely damaged, the dentist will perform a restoration using a crown; and if the nerve is affected, a root canal must be performed. Untreated caries continue to progress and bacteria eventually attack the alveolar bone under the root, leading to a painful abscess that must be treated with antibiotics and then proceed to mechanical treatment. Antibiotics are also used in the treatment of periodontal disease in combination with scaling and root planing, curettage, dental surgery or as a lone treatment to reduce bacteria before and/or after these common periodontal procedures.

The intensive use of antibiotics has introduced a selection pressure leading to the concerning development of populations of antibiotic-resistant microorganisms and to a general decrease in therapeutic efficacy. These resistances, which were initially sporadic, have become massive and worrying. Some bacterial strains have become multiresistant, i.e., resistant to several antibiotics, and other strains have become totally resistant, i.e., resistant to all available antibiotics. Fortunately, the latter case is still rare, but the phenomenon is increasing and puts physicians in a therapeutic impasse. Thus, in the current context of increasing and worrying antibiotic resistance, scientists and health professionals are seeking to identify new therapeutic strategies, including in the treatment and/or prevention of infectious diseases of the oral cavity.

### SUMMARY

The present Inventors have found, surprisingly, that Saccharomyces cerevisiae var. boulardii yeast strains have an increased ability, compared with other probiotics, to inhibit the growth of periodontal and cariogenic agents. One of these strains is, for example, the yeast strain Saccharomyces cerevisiae var. boulardii deposited, by the Applicant, on Aug. 21, 2007 at the CNCM (Collection Nationale de Cultures de Microorganismes, 25 rue du Docteur Roux, 75724 Paris Cedex 15, France) under number I-3799.

The present Inventors have also demonstrated a synergy between a Saccharomyces cerevisiae var. boulardii yeast strain and a Saccharomyces cerevisiae yeast strain in dry and inactivated form, to significantly inhibit the growth of certain periodontal and cariogenic pathogens. One such Saccharomyces cerevisiae var. boulardii yeast strain is, for example, strain number I-3799 already described above. Such a Saccharomyces cerevisiae yeast strain is, for example, the Saccharomyces cerevisiae strain deposited, by the Applicant, on Oct. 17, 2007 at the CNCM under number I-3856.

The object of the present invention is thus a Saccharomyces cerevisiae var. boulardii yeast strain, or its combination with the inactivated dry form of a Saccharomyces cerevisiae yeast strain, for use in the prevention and/or treatment of an infectious disease of the oral cavity in a subject.

In certain embodiments, the Saccharomyces cerevisiae var. boulardii yeast is in a dry live form.

In certain embodiments, the Saccharomyces cerevisiae var. boulardii yeast strain is the strain deposited on Aug. 21, 2007 with the CNCM under number I-3799.

In certain embodiments, the yeast strain Saccharomyces cerevisiae is the strain deposited on Oct. 17, 2007 with the CNCM under number I-3856.

The present invention also relates to a dietary supplement comprising a Saccharomyces cerevisiae var. boulardii yeast strain, or its combination with the inactivated dry form of a Saccharomyces cerevisiae yeast strain, as defined above, for use in the prevention and/or treatment of an infectious disease of the oral cavity in a subject.

In certain embodiments, the dietary supplement is in the form of a lozenge to be sucked, a candy, a chewing gum, an orodispersible powder or a powder to be diluted in water in the form of a stick or sachet, a tablet to be sucked or chewed, a gum, a capsule, a tablet, drops, or a vial with a measuring cap.

The present invention also relates to a parapharmaceutical or cosmetic composition comprising a Saccharomyces cerevisiae var. boulardii yeast strain, or its combination with the inactivated dry form of a Saccharomyces cerevisiae yeast strain, as defined above, for use in the prevention and/or treatment of an infectious disease of the oral cavity in a subject.

In certain embodiments, the parapharmaceutical or cosmetic composition is in the form of a toothpaste, a mouthwash, an oral spray, an oral cream or gel, an orodispersible sheet, a powder to be sprinkled directly into the oral cavity, an orodispersible powder or a powder to be diluted in water in stick or sachet form, or a vial with a measuring cap.

The present invention further relates to a pharmaceutical composition for use in the prevention and/or treatment of an infectious disease of the oral cavity in a subject, the pharmaceutical composition comprising an effective amount of a Saccharomyces cerevisiae var. boulardii yeast strain, or its combination with the inactivated dry form of a Saccharomyces cerevisiae yeast strain, as defined above, and at least one physiologically acceptable excipient.

In certain embodiments, the pharmaceutical composition is intended for topical administration or oral administration.

In certain embodiments, the pharmaceutical composition further comprises at least one additional pharmaceutical active principle having soothing, anti-irritant, analgesic, antalgic, anti-inflammatory, healing, antibiotic, antipyretic, or antifungal activity.

The present invention also relates to a dental medical device comprising a Saccharomyces cerevisiae var. boulardii yeast strain, or its combination with the inactivated dry form of a Saccharomyces cerevisiae yeast strain, as defined above, for use in the prevention and/or treatment of an infectious disease of the oral cavity in a subject.

In certain embodiments, the dental device is a dental implant, a dental crown, a dental bridge, a dental onlay, or a dental prosthesis.

In certain embodiments, the infectious disease to be prevented or treated according to the present invention is dental caries, gingivitis or periodontitis.

In certain embodiments, the infectious disease to be prevented or treated according to the present invention is a side effect of a medical treatment or is present or likely to develop or recur in a patient suffering from an immunodeficiency, or is present in a pregnant woman, an elderly person, a child, or a patient with a hyperinflammatory phenotype.

A more detailed description of certain preferred embodiments of the invention is given below.

### BRIEF DESCRIPTION OF THE DRAWINGS

FIG. 1: Halo results obtained (A) with Saccharomyces cerevisiae strain number I-3856, in the presence of the pathogenic species: Fusobacterium nucleatum (Fn), Prevotella intermedia (Pi), Aggregatibacter actinomycetemcomitans (Aa), Porphyromonas gingivalis (Pg) (B) with Saccharomyces cerevisiae var. boulardii strain number I-3799, in the presence of the pathogenic species: Fusobacterium nucleatum (Fn), Prevotella intermedia (Pi), Aggregatibacter actinomycetemcomitans (Aa), and Porphyromonas gingivalis (Pg); (a) under aerobic conditions or (b) under anaerobic conditions.

FIG. 2: Effects of Saccharomyces cerevisiae strain number 1-3856 (1-3856); Saccharomyces cerevisiae var. boulardii strain number I-3799 (Boulardii); and a control (Control) on different bacterial species in a 14-species biofilm, including 4 periodontopathogens (A to D), and 2 cariopathogens (E and F): (A) Aggregatibacter actinomycetemcomitans (Aa), (B) Fusobacterium nucleatum (Fn), (C) Porphyromonas gingivalis (Pg), (D) Prevotella intermedia (Pi), (E) Streptococcus mutans (Sm), (F) Streptococcus sobrinus. Values represent mean±SE from triplicates (N=3). *p<0.05 vs. Control; **p<0.01 vs. Control; ***p<0.001 vs. Control; ****p<0.0001 vs. Control.

FIG. 3: Effects of the inactivated dry form of Saccharomyces cerevisiae strain number 1-3856 (IY)); Saccharomyces cerevisiae var. boulardii strain number 1-3799 (Boulardii); the combination of the inactivated dry form of Saccharomyces cerevisiae strain number 1-3856 and Saccharomyces cerevisiae var. boulardii strain number 1-3799 (IY+Boulardii); and a control on different bacterial species of a 14-species biofilm, including 4 periodontopathogens (A to D), and 2 cariopathogens (E and F): (A) Aggregatibacter actinomycetemcomitans (Aa), (B) Fusobacterium nucleatum (Fn), (C) Porphyromonas gingivalis (Pg), (D) Prevotella intermedia (Pi), (E) Streptococcus mutans (Sm), (F) Streptococcus sobrinus. Values represent mean±SE from triplicates (N=3). *p<0.05 vs. Control; **p<0.01 vs. Control; ***p<0.001 vs. Control; ****p<0.0001 vs. Control; #p<0.05 vs. Nutri; $ p<0.05 vs. Boulardii.

FIG. 4: Dose-response curves of Saccharomyces cerevisiae var. boulardii CNCM I-3799 yeast on the secretion of the following different inflammatory parameters: (A) IL-6, (B) IL-8, (C) IL-10, (D) MCP-1, (E) PGE-2, and (F) isoprostane, by primary human monocytes after LPS challenge. Values are expressed as mean±SE from duplicate experiments performed for three donors (n=6). ***p<0.001 vs. Control, ****p<0.0001 vs. Control.

FIG. 5: Dose-response curves of Saccharomyces cerevisiae var. boulardii CNCM I-3799 yeast on the secretion of the following different inflammatory parameters: (A) IL-6, (B) IL-8, (C) PGE-2, and (D) isoprostane, by primary human gingival fibroblasts. Values are expressed as mean±SE for three experiments (n=3). ***p<0.001 vs. Control, **** p<0.0001 vs. Control.

### DETAILED DESCRIPTION

As mentioned above, the present invention relates to a Saccharomyces cerevisiae var. boulardii yeast strain having the ability to inhibit the growth of periodontopathogenic and cariogenic agents and the use thereof, alone or in combination with a dry and inactivated Saccharomyces cerevisiae yeast, in the treatment and/or prevention of infectious diseases of the oral cavity.

I—Saccharomyces cerevisiae var. boulardii Yeast Strain

The expression “yeast strain” refers to a relatively homogeneous population of yeast cells. A yeast strain is obtained from a clone, a clone being a population of cells obtained from a single yeast cell.

A Saccharomyces cerevisiae var. boulardii yeast strain that can be used in the context of the present invention is, for example, the strain that was deposited on Aug. 21, 2007, by the present Applicant, at the CNCM (Collection Nationale de Cultures de Microorganismes, 25 rue du Docteur Roux, 75724 Paris Cedex 15, France) under number I-3799.

This Saccharomyces cerevisiae var. boulardii strain has previously been described by the present Applicant itself in WO 2009/103884, where it is presented as being useful in the prevention and/or treatment of pathologies, disorders or ailments of the intestine.

In the context of the present invention, a Saccharomyces cerevisiae var. boulardii yeast strain is in the form of yeast cells obtained by culturing (or multiplying) the starting strain. A Saccharomyces cerevisiae var. boulardii strain can be cultured by any suitable method. The processes for culturing a Saccharomyces cerevisiae var. boulardii strain are known in the art, and the skilled person knows how to optimize the culture conditions for each strain according to its nature. The yeast cells can for example be obtained by multiplying a Saccharomyces cerevisiae var. boulardii strain in a culture medium as described in the reference book “Yeast Technology”, 2^{nd} Edition, 1991, Reed and Nagodawithana, published by Van Nostrand Reinhold (ISBN 0-442-31892-8).

Thus, for example, on an industrial scale, the Saccharomyces cerevisiae var. boulardii yeast that can be used in the context of the present invention can be obtained by a process comprising the following steps:
culturing a Saccharomyces cerevisiae var. boulardii strain (for example strain number I-3799) in a culture medium in several steps, first under semi-anaerobic conditions and then under aerobic conditions (oxygen-rich medium/atmosphere) to obtain a multiplication of the starting yeast cells;
and separating, by centrifugation, the yeast cells thus produced to obtain a liquid yeast cream containing between 12% and 25% of yeast dry matter.

The Saccharomyces cerevisiae var. boulardii yeast used in the context of the invention is in the form of live yeast. The term “live yeast”, which is synonymous with “active yeast”, refers to a population of yeast cells that are metabolically active.

The live yeast used in the context of the present invention is in the form of a dry yeast. A dry yeast is characterized by a low water content, and generally comprises a yeast dry matter content greater than 90%, preferably a dry matter content comprised between 92% and 98%, for example a dry matter content comprised between 94.5% and 96.5%. One of the advantages of dry yeast is its long storage life.

Thus, the process for producing Saccharomyces cerevisiae var. boulardii yeast can also include a subsequent drying step, to obtain a yeast in dry form. This drying step may or may not be followed by a grinding operation. The drying may be, for example, freeze-drying, fluidized bed drying, drum drying or spray drying. In the case of freeze-drying, a maltodextrin or lactose type carrier can be used.

II—Combination of the Saccharomyces cerevisiae var. boulardii Yeast Strain and the Dry Inactivated Saccharomyces cerevisiae Yeast Strain

As indicated above, the present Inventors have demonstrated a synergy between a Saccharomyces cerevisiae var. boulardii yeast strain (for example Saccharomyces cerevisiae var. boulardii yeast strain number I-3799) and a Saccharomyces cerevisiae yeast strain in dry inactivated form to significantly inhibit the growth of periodontogenic and cariogenic bacteria. The terms “synergy” and “synergistic action”, which are used interchangeably herein, refer to a coordinated action of the two yeast strains creating an effect greater than the sum of the effects expected if they had acted independently, or creating an effect that each of them could not have achieved by acting in isolation.

The Saccharomyces cerevisiae yeast used in combination with a Saccharomyces cerevisiae var. boulardii yeast is a dry inactivated yeast. The terms “inactivated yeast” and “deactivated yeast”, which are used interchangeably herein, refer to a yeast that is no longer alive, i.e., a yeast whose metabolism has been irreversibly stopped. An inactivated yeast according to the invention can be obtained by any suitable method. Suitable techniques well known to the skilled person include heat treatment of the yeast, spray treatment, drum drying or any combination of these treatments. Inactivated yeast is generally found in dry form. A dry yeast is characterized by a low water content, and generally comprises a yeast dry matter content greater than 90%, preferably a dry matter content comprised between 92% and 98%, for example a dry matter content comprised between 94.5% and 96.5%. One of the advantages of dry yeast is its long storage life.

The Saccharomyces cerevisiae var. boulardii yeast strain used in the combination can be any Saccharomyces cerevisiae var. boulardii yeast strain.

The dry inactivated Saccharomyces cerevisiae yeast strain used in the combination can be any Saccharomyces cerevisiae strain.

In certain particular embodiments, the Saccharomyces cerevisiae yeast strain, used in inactivated dry form in the context of the invention in combination with a Saccharomyces cerevisiae var. boulardii strain, is the Saccharomyces cerevisiae strain deposited, on Oct. 17, 2007, at the CNCM under number 1-3856. This Saccharomyces cerevisiae strain has been previously described, by the present Applicant itself, in WO 2009/103884, where it is presented as being useful in the prevention and/or treatment of pathologies, disorders or ailments of the intestine; and in WO 2014/009656, where it is presented as being effective in controlling vaginal Candida overgrowth and in preventing the recurrence of vaginal or vulvovaginal candidiasis.

In certain particular embodiments, the Saccharomyces cerevisiae var. boulardii yeast strain, used in the context of the invention in combination with the Saccharomyces cerevisiae yeast strain in inactivated dry form, is the Saccharomyces cerevisiae var. boulardii yeast strain deposited with the CNCM on Aug. 21, 2007 under number I-3799.

The term “combination”, as used herein, refers to a mixture of Saccharomyces cerevisiae var. boulardii (for example strain number I-3799) and Saccharomyces cerevisiae in inactivated dry form (for example strain number I-3856). The mixture can be prepared in any proportions. Thus, preferably, the combination comprises a ratio of Saccharomyces cerevisiae var. boulardii I-3799 to Saccharomyces cerevisiae in inactivated dry form ranging from about 90:10 (w/w=weight/weight) to about 10:90 (w/w). Thus, the ratio of Saccharomyces cerevisiae var. boulardii I-3799 to Saccharomyces cerevisiae in inactivated dry form may, for example, be about 90:10 (w/w), about 80:20 (w/w), about 70:30 (w/w), about 60:40 (w/w), about 50:50 (w/w), about 40:60 (w/w), about 30:70 (w/w), about 20:80 (w/w) or about 10:90 (w/w). The term “about”, as used herein in reference to a number, generally includes numbers that fall within a 10% range in either direction of the number (greater or less than the number), except in the case where that number exceeds 100% of a possible value.

In certain particular embodiments of the invention, the ratio of Saccharomyces cerevisiae var. boulardii I-3799 to Saccharomyces cerevisiae I-3856 in inactivated dry form is about 50:50 (w/w).

III—Uses of the Saccharomyces cerevisiae var. boulardii Yeast Strain and the Combination in the Treatment and/or Prevention of Infectious Diseases of the Oral Cavity

The invention thus relates to a Saccharomyces cerevisiae var. boulardii yeast strain (for example strain number I-3799) or its combination with the inactivated dry form of a Saccharomyces cerevisiae strain (for example strain number I-3856), for the treatment and/or prevention of infectious diseases of the oral cavity. The invention also relates to a method for treating and/or preventing an infectious disease of the oral cavity in a subject, the method comprising a step of administering to the subject an effective amount of a Saccharomyces cerevisiae strain (for example strain number I-3799), or its combination with the inactivated dry form of a Saccharomyces cerevisiae strain (for example strain number I-3856). The invention also relates to the use of a Saccharomyces cerevisiae var. boulardii strain (for example strain number I-3799), or its combination with the inactivated dry form of a Saccharomyces cerevisiae strain (for example strain number I-3856), for the manufacture of a medicinal product for the treatment and/or prevention of infectious diseases of the oral cavity.

In the context of the present invention, “treatment” means a method whose aim is to: (1) delay or prevent the onset of a disease or clinical condition; (2) slow or halt the progression, worsening or deterioration of disease symptoms; (3) provide improvements in disease symptoms; and/or (4) cure the disease. A treatment may be administered before the onset of the disease, for prophylactic action (referred to as “prevention”), or it may be administered after the initiation of the disease, for therapeutic action.

The term “subject” here refers to a mammal, and more particularly a human being, which has the potential to become a victim of an oral infection but which does not necessarily have such an infection. The term “subject” does not refer to a particular age and therefore includes newborns, children, adolescents, adults, and the elderly. The term “patient” is sometimes used here, instead of “subject”, when the subject is suffering (i.e., has been diagnosed as suffering) from an infectious disease of the oral cavity.

The term “infectious disease of the oral cavity” refers to a localized medical condition in the mouth that results from infection by a pathogenic microorganism. Infectious diseases of the oral cavity include dental caries and periodontal disease.

The term “dental caries” or “caries” refers to an infectious disease of the tooth that causes damage to the enamel, the dentin, and/or the pulp/cement. Cariogenic bacteria, i.e., bacteria that promote the development of caries, include: Streptococcus mutans (which promotes smooth and proximal surface caries); Streptococcus mutans and lactobacilli (which promote fissure caries); Actinomycetes viscosus and Actinomycetes naeslundi (which promote root caries and colonization of the dentin and root); and lactobacilli (which promote colonization of the dentin and root). Tooth colonization begins with streptococci, then actinomycetes and, finally, lactobacilli. The bacterial predominance varies with the depth of the lesion. Mutans streptococci is associated with initial lesions, whereas lactobacilli appear later in a deep lesion. The following pathogens are also known to be associated with the occurrence of dental caries: Streptococcus sobrinus and Lactobacilles.

The term “periodontal diseases” means diseases and pathological conditions that affect the supporting tissues of the teeth, i.e., the bone and the gum. These are infections caused by the accumulation of pathogenic bacteria and their toxins on the gum line around the teeth. The main periodontal diseases are gingivitis and periodontitis. Gingivitis is the inflammation of the gums around the teeth. This condition is reversible when treated. Periodontitis, on the other hand, is a more severe pathology which deteriorates the gum and the bone supporting the teeth. The following bacteria are for example also known to be associated with the occurrence of periodontal disease: Porphyromonas gingivalis, Treponema denticola, Tannerella forsythensis, Aggregatibacter actinomycetemcomitans, Actinobacillus actinomycetemcomitans, Actinomycetes odontolyticus, Actinomycetes naeslundii, Fusobacterium Nucleatum, Campylobacter rectus, Peptostreptococcus micros, Prevotella melaninogenica, Prevotella intermedia, a, Capnocytophaga, Eikenella, Bacteroidetes, Eubacterium saphenum, P. endodontalis, Prevotella denticola, Parvimonas micra, Filifactor alocis, Desulfobulus species and Synergistetes species.

According to the meta-analysis by Guerra et al., 600 bacterial species have been identified in the human oral microbiome. However, 53% of the species in the oral microbiome have not yet been identified and 35% are not cultivable.

A method for treating and/or preventing an infectious disease of the oral cavity according to the invention comprises administering to a subject an effective amount of a Saccharomyces cerevisiae var. boulardii strain (for example strain number I-3799), or its combination with the inactivated dry form of the Saccharomyces cerevisiae strain (for example strain number I-3856). The effective amount, which may be administered in one or more doses, can be determined by the physician or dentist. The exact amount to be administered may vary from patient to patient, depending on the patient's age, weight and general condition, the severity and/or extent of dental caries or periodontal disease, etc. The effective amount to be administered may also vary depending on the desired therapeutic effect (i.e., prevention of infectious disease of the oral cavity or treatment of infectious disease of the oral cavity).

Preferably, the administration of Saccharomyces cerevisiae var. boulardii (for example strain number 1-3799), or its combination with the inactivated dry form of Saccharomyces cerevisiae (for example strain number I-3856), is an oral administration with a localized effect to the oral cavity, however, in certain embodiments, the yeast (or combination) may be swallowed (see below).

For example, the dose of the combination of a Saccharomyces cerevisiae var. boulardii yeast in dry form (for example strain number 1-3799) and a Saccharomyces cerevisiae yeast in inactivated dry form (for example strain number 1-3856) may be comprised between 1 mg and 10 g, preferably between 100 mg and 5 g of the mixture.

The recommended concentration of live Saccharomyces cerevisiae var. boulardii yeast can be comprised between 1·10⁷ and 1·10¹¹ CFU/g, and preferably between 5·10⁸ and 1·10¹⁰ CFU/g.

A treatment method according to the invention can be used to treat a first episode of infectious disease of the oral cavity, in particular gingivitis or periodontitis, or a recurrence. In either case, the patient may have previously been treated with a standard antibiotic. Alternatively, the Saccharomyces cerevisiae var. boulardii (for example strain number I-3799), or its combination with a Saccharomyces cerevisiae yeast (for example strain number I-3856), may be the first treatment prescribed to the patient.

A treatment method according to the invention can also be used to prevent an infectious disease of the oral cavity in a patient, whether the infectious disease of the oral cavity is a first episode or a recurrence. This may be the case for patients receiving medical treatment known to cause periodontal disturbances, such as immunosuppressants, phenytoin (anti-epileptic), calcium channel blockers such as nifedipine (which are indicated for the treatment of various heart conditions such as angina, arrhythmias, and high blood pressure). This may also be the case in patients with immune vulnerability, for example, associated with diseases such as Sjögren's syndrome, HIV infection, uncontrolled diabetes, certain endocrine disorders, malnutrition or malabsorption, such as vitamin B deficiency. This can also be the case for patients with reduced salivary flow (for example certain elderly people). It can also be the case of smokers or people under stress. It can also be the case of pregnant women for whom the prevention of oral diseases can begin at the beginning of pregnancy. Finally, it can be the case of people with a hyperinflammatory phenotype, i.e., with a propensity to provoke an exacerbated inflammatory reaction by responding, to an aggression of the same intensity, with an overproduction of pro-inflammatory cytokines (PGE2, IL-1, TNF), factors that play a major role in periodontal destruction.

In certain embodiments, a treatment according to the invention is administered alone. In other words, the Saccharomyces cerevisiae var. boulardii yeast (for example strain number I-3799), or its combination with the inactivated dry form of a Saccharomyces cerevisiae yeast (for example strain number I-3856), is the only agent to be administered, with the exception of possible antiseptic mouthwashes.

In other embodiments, a treatment according to the invention is administered in combination with another therapy, for example with the administration of a standard antibiotic or antiseptic, and/or with a mechanical or surgical procedure, such as scaling, root planing, curettage, filling, restoration, or root canal.

### IV—Dietary Supplements, Cosmetic and Pharmaceutical Compositions

As indicated above, the Saccharomyces cerevisiae var. boulardii strain (for example strain number I-3799), or its combination with the inactivated dry form of a Saccharomyces cerevisiae strain (for example strain number I-3856), may be administered as such or as a preparation or composition.

Thus, in certain embodiments, the Saccharomyces cerevisiae var. boulardii strain (for example strain number I-3799), or its combination with the inactivated dry form of a Saccharomyces cerevisiae strain (for example strain number I-3856), is present in a dietary supplement in the form of a lozenge to be sucked, a candy, a chewing gum, an orodispersible powder or a powder to be diluted in water in the form of a stick or sachet, a tablet to be sucked or chewed, a gum, a capsule, a tablet, drops, or a vial with a measuring cap, etc.

In other embodiments, the Saccharomyces cerevisiae var. boulardii strain (for example strain number I-3799), or its combination with the inactivated dry form of a Saccharomyces cerevisiae strain (for example strain number I-3856), is present in a parapharmaceutical (or cosmetic) composition in the form of a toothpaste, a mouthwash, an oral spray, a cream or an oral gel, an orodispersible sheet, or a powder which can be sprinkled directly into the oral cavity, an orodispersible powder or a powder to be diluted in water in the form of a stick or a sachet, a vial with a measuring cap, etc.

In still other embodiments, the Saccharomyces cerevisiae var. boulardii strain (for example strain number I-3799), or its combination with the inactivated dry form of a Saccharomyces cerevisiae strain (for example strain number I-3856), is present in a pharmaceutical composition together with at least one physiologically acceptable excipient. Thus, more specifically, a pharmaceutical composition according to the invention comprises an effective amount of the Saccharomyces cerevisiae var. boulardii yeast strain (for example strain number I-3799), or its combination with the inactivated dry form of a Saccharomyces cerevisiae strain (for example strain number I-3856), and at least one physiologically acceptable excipient. A pharmaceutical composition according to the invention may be classified as a pharmaceutical preparation available by prescription or over the counter.

In the context of the present invention, “physiologically acceptable excipient” is understood to mean any medium or additive that does not interfere with the efficacy of the biological activity of the active principle (here, the Saccharomyces cerevisiae var. boulardii strain or its combination), and that is not excessively toxic to the patient or subject at the concentrations at which it is administered. A physiologically acceptable excipient may be an excipient suitable for administration to mammals, particularly to humans.

The pharmaceutical compositions according to the present invention can be administered using any combination of dosage and route of administration effective to achieve the desired therapeutic/prophylactic effect. As already indicated above, the exact amount to be administered may vary from patient to patient, depending on the patient's age, weight and general condition, the severity and extent of the oral infection and the nature of the associated oral disease (dental caries, gingivitis, periodontitis), etc. The route of administration (topical or systemic) can be chosen according to the severity and extent of the bacterial infection and/or according to the patient's age and/or health.

By way of example, the object of the invention is a pharmaceutical composition as defined above for use of a combination of the Saccharomyces cerevisiae var. boulardii yeast (for example strain number I-3799) and the inactivated dry form of a Saccharomyces cerevisiae yeast (for example strain number I-3856) in an amount comprised between 1 mg and 10 g, preferably between 100 mg and 5 g, which amount may be administered one or more times a day.

The recommended concentration of Saccharomyces cerevisiae var. boulardii yeast can be comprised between 1·10⁷ and 1·10¹¹ CFU/g, and preferably between 5·10⁸ and 1·10¹⁰ CFU/g.

The formulation of a pharmaceutical composition according to the present invention may vary depending on the route of administration and dosage for which the composition is intended to be used. After formulation with at least one physiologically acceptable excipient, a pharmaceutical composition of the invention may be in any form suitable for administration to a human being, for example in the form of tablets, sugar-coated pills, capsules, drops, syrups, emulsions, ointments, pastes, gels, powders, sachets, injectable solutions, etc. The person skilled in the art knows how to select the most appropriate carriers and excipients for the preparation of a given type of formulation. A composition according to the invention may further comprise additives such as preservatives, sweeteners, flavors, viscosity agents, dyes, humectants, disintegrants, absorption accelerators, lubricants, etc.

In certain embodiments, a pharmaceutical composition according to the invention contains only one active agent: the Saccharomyces cerevisiae var. boulardii yeast (for example strain number 1-3799) or its combination with the inactivated dry form of a Saccharomyces cerevisiae yeast (for example strain number I-3856). Such a pharmaceutical composition does not contain, in particular, another living microorganism or a combination of living microorganisms.

In other embodiments, a pharmaceutical composition according to the invention further contains at least one additional pharmaceutical active principle (i.e., in addition to the Saccharomyces cerevisiae var. boulardii yeast or yeasts of the combination). “Pharmaceutical active principle” is understood to mean any compound or substance whose administration has a therapeutic effect or whose administration has a beneficial effect on the health or general condition of a patient or subject to whom it is administered.

Thus, a pharmaceutical active principle may be active against a bacterial infection of the oral cavity that is to be prevented or treated by administration of the pharmaceutical composition; or may be active against a condition or symptom associated with the infectious disease of the oral cavity (for example pain or fever or halitosis); or may increase the availability and/or activity of the active principle(s) of the pharmaceutical composition.

Examples of active pharmaceutical principles that may be present in a composition of the present invention include, but are not limited to, active principles having soothing, anti-irritant, analgesic, antalgic, anti-inflammatory, healing, antibiotic, antipyretic or antifungal activity (without effect on the Saccharomyces cerevisiae var. boulardii yeast or on its combination with a Saccharomyces cerevisiae yeast in inactivated dry form), etc.

In still other embodiments, the Saccharomyces cerevisiae var. boulardii strain (for example strain number I-3799), or its combination with the inactivated dry form of a Saccharomyces cerevisiae strain (for example strain number I-3856), is present on/in a dental medical device: for example a dental implant, a dental crown, a dental bridge, a dental onlay, a dental prosthesis, etc.

Unless otherwise defined, all technical and scientific terms used in the Description have the same meaning as commonly understood by an ordinary person skilled in the field to which this invention belongs. Similarly, all publications, patent applications, patents and other references mentioned herein are incorporated by reference.

### Examples

The following examples describe certain embodiments of the present invention. However, it is understood that the examples and figures are provided by way of illustration only and in no way limit the scope of the invention.

### Effects of Different Probiotics on Periodontopathogenic Bacteria and Cariogenic Bacteria

### A. Probiotics Tested and Periodontopathogenic or Cariogenic Bacteria

Yeast Products. In the studies described below, two live dry yeasts and one inactivated dry yeast were evaluated.

The two live yeasts are:
Saccharomyces cerevisiae strain number CNCM I-3856 (deposited by the Applicant at the CNCM on Oct. 17, 2007), and
Saccharomyces cerevisiae var. boulardii strain number CNCM I-3799 (already described above).

The inactivated dry yeast is:
The inactivated dry form of Saccharomyces cerevisiae strain number I-3856 (already described above).

### Composition of a 14-Species Complex Biofilm:

Periodontopathogenic Bacteria and Cariogenic Bacteria. The periodontopathogenic bacteria used include:
Prevotella intermedia (formerly known as Bacteroides intermedius) which is an obligate anaerobic, Gram-negative pathogenic bacterium involved in periodontal infections, including gingivitis and periodontitis, and often found in acute necrotizing ulcerative gingivitis;
Porphyromonas gingivalis, which is an anerobic, non-motile, Gram-negative, rod-shaped pathogenic bacterium found in the oral cavity, where it is involved in some forms of periodontal disease;
Fusobacterium nucleatum, which is an anerobic, invasive, adherent and inflammatory bacterium, foreign to the human oral cavity, and which plays a role in periodontal disease by being a key component of periodontal plaque due to its abundance and ability to associate with other species in the oral cavity; and
Aggregatibacter actinomycetemcomitans, which is a small, slow-growing, facultative aero-anaerobic, Gram-negative bacterium belonging to the physiological oral flora of humans and playing a role in periodontal disease.

The cariogenic bacteria used include:
Streptococcus mutans, which is a Gram-positive cocci bacterium, part of the commensal flora of the oral cavity where it is responsible for dental caries in combination with dietary sugar, which it transforms into lactic acid; and
Streptococcus sobrinus, which is a Gram-negative, non-motile, anaerobic bacterium, present in large quantities in the dental plaque of patients with caries.

Commensal Bacteria of the Oral cavity. The commensal bacteria of the oral cavity used in the present study include: Streptococcus sanguinis; Streptococcus gordonii; Streptococcus salivarius; Streptococcus mitis; Streptococcus oralis; Actinomyces viscosus; Actinomyces naeslundii; and Veillonella parvula.

B. Antibacterial Activity of Yeasts against 6 Pathogenic Species

Protocol. The halo technique was used to detect and quantify the inhibition rate of commensal and pathogenic bacteria on agar plates. This technique is a competitive test, which consists in inoculating two spots, close to each other and each containing a different bacterial species, on an agar plate. Then, the ability of one to inhibit the growth of the other can be evaluated.

The agar plates were seeded with the culture of one of the 2 yeasts (live) adjusted to an optical density at 600 nm (OD₆₀₀) of 0.5, which corresponds to a concentration of approximately 10⁸ CFU/mL—the BHI-2 agar plates did not contain hemin, menadione or blood. Each spot contained 7 μL of the culture of one of the 2 live yeasts of concentration 10⁸ CFU/mL.

After 24 hours of aerobic or anaerobic incubation, the agar plates were again inoculated for 24 h with 7 μL of a culture of a pathogenic species (Fusobacterium nucleatum (Fn), Prevotella intermedia (Pi), Aggregatibacter actinomycetemcomitans (Aa), and Porphyromonas gingivalis (Pg)), in close proximity to the spot containing the yeast species.

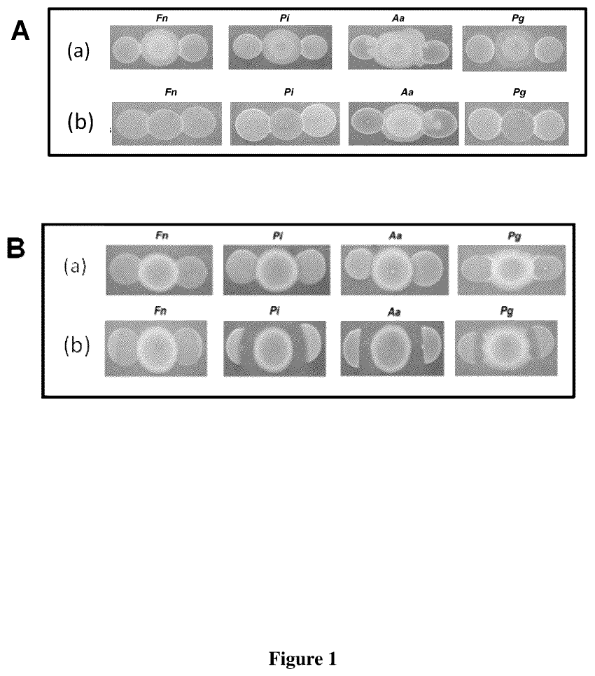

In total, the agar plates were incubated for 48 hours under anaerobic or aerobic conditions. After the 48-hour incubation, the inhibition surface was inspected and calibrated (with a ruler) (Table 1), and a standardized photograph (distance between the agar plate and the camera) was taken of each of the agar plates (FIG. 1).

The pathogens tested with the 2 live yeasts were the following: Fusobacterium nucleatum (Fn), Prevotella intermedia (Pi), Aggregatibacter actinomycetemcomitans (Aa), and Porphyromonas gingivalis (Pg),

Results. The results obtained are presented in FIG. 1 and summarized in Table 1.

TABLE 1
Rate of inhibition (mean ± standard deviation, N = 3)
induced by the yeast species Saccharomyces cerevisiae var.
boulardii I-3799 on the growth of certain oral pathogenic bacteria.

| | inhibition distance |
|---|---|
| Bacterial Competition | mean ± SD* (mm) |
| S. boulardii (24 h anaerobic) + P. intermedia | 6.19 ± 0.09 |
| (24 h anaerobic) | |
| S. boulardii (24 h anaerobic) + P. gingivalis | 3.11 ± 0.14 |
| (24 h anaerobic) | |
| S. boulardii (24 h anaerobic) + A. | 4.48 ± 0.14 |
| actinomycetemcomitans (24 h aerobic) | |
| S. boulardii (24 h aerobic) + A. | 0.52 ± 0.07 |
| actinomycetemcomitans (24 h aerobic) | |

*SD = standard deviation

Table 1 provides the measured inhibition distances for cases where the growth of certain oral pathogens was significantly inhibited.

The results obtained show that the Saccharomyces cerevisiae var. boulardii strain induces a significant inhibition of the growth of the pathogenic species Prevotella intermedia, Aggregatibacter actinomycetemcomitans and Porphyromonas gingivalis under anaerobic conditions.

In addition, the growth of Aggregatibacter actinomycetemcomitans was also inhibited by the Saccharomyces cerevisiae var. boulardii strain under aerobic conditions.

Table 1. shows that the greatest order of magnitude of inhibition was observed on the growth of Prevotella intermedia under anaerobic conditions compared with other bacteria and pathogens.

It is also observed in FIG. 1 that the Saccharomyces cerevisiae CNCM I-3856 strain showed no significant inhibition of the pathogenic species.

Conclusion. Only the Saccharomyces cerevisiae var. boulardii strain inhibited the growth of oral pathogens on agar plates.

### C. Evaluation of the Effect of Yeast Products on the Composition of a Complex Biofilm Comprising 14 Species.

### 1) Effects of Live Yeast Studied

Bioreactor Protocol. A multispecies community was established in a BIOSTAT B TWIN® reactor (Sartorius, Germany). A medium containing 750 mL of BHI-2 (Brain Heart Infusion (BHI) supplemented with 2.5 g/L mucin, 1.0 g/L yeast extract, 0.1 g/L cysteine, 2.0 g/L sodium bicarbonate and 0.25% (v/v) glutamic acid) was added to the bioreactor along with 5.0 mg/mL hemin, 1.0 mg/mL menadione and 200 μL/L Antifoam Y-30 (Sigma, St. Louis, USA). The mixture was pre-reduced for 24 hours at 37° C. by bubbling 100% nitrogen (N₂) and 5% carbon dioxide (CO₂) into the mixture while stirring continuously at 300 rpm, at a pH set at 6.7±0.1. After 24 hours, cultures of Streptococcus mutans (E), Streptococcus sobrinus (F), Prevotella intermedia (D), Porphyromonas gingivalis (C), Fusobacterium nucleatum (B), and Aggregatibacter actinomycetemcomitans (A) were added at an optical density of 1.4 and added to the bioreactor mixture. The medium was not replaced for the first 48 hours and then was replaced at a rate of 200 mL/24 hours.

Culture Protocol. Cultures of Saccharomyces cerevisiae strain number I-3856 (I-3856) and Saccharomyces cerevisiae var. boulardii strain number 1-3799 (Boulardii) (performed overnight) were adjusted to an optical density at 600 nm (OD₆₀₀) of 0.5 (˜1·10⁸ CFU/mL) in BHI-2 (see composition above). Aliquots of 1800 μL of these adjusted cultures were inoculated onto 24-well plates containing hydroxyapatite discs at the bottom of each well. 200 μL of the 14-species biofilm mixture from the bioreactor was added to each well. The controls contained 200 μL of the 14-species samples and 1800 μL of BHI-2. The 24-well plates were incubated under micro-aerobic conditions (6% oxygen) for 24 hours. Then, the supernatants were removed and the biofilms attached to the hydroxyapatite discs were washed with phosphate buffered saline (PBS). The biofilms were detached with 1500 μL of 0.05% Trypsin-EDTA for 45 minutes at 37° C. and 230 rpm, transferred to Eppendorf tubes, and centrifuged (6010×g, for 5 minutes). After removing the trypsin, the biofilm pellet was re-suspended in 500 μL of PBS, and DNA extraction was performed according to Qiagen instructions and analyzed by qPCR.

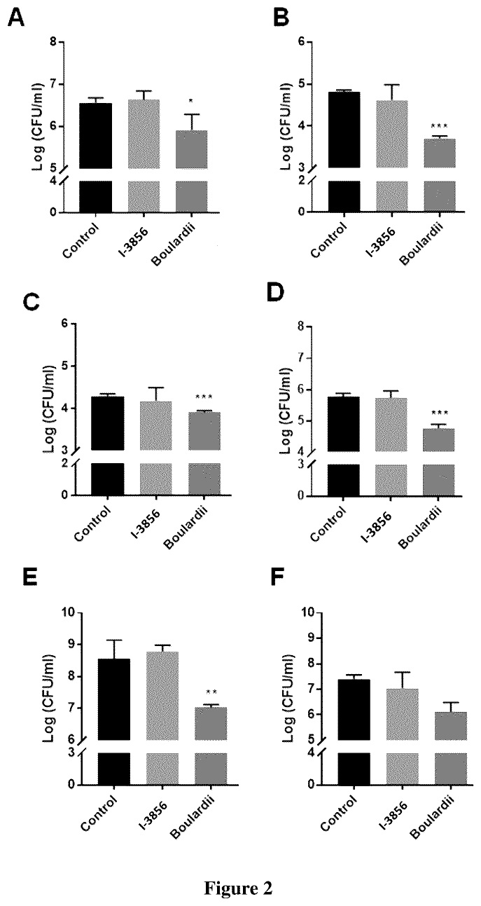

Results Obtained on Live Yeast. The results obtained are presented in FIG. 2 (A-F).

It is observed that the Saccharomyces cerevisiae var. boulardii strain is able to significantly inhibit the following periodontopathogens: Aggregatibacter actinomycetemcomitans (A), Fusobacterium nucleatum (B), Porphyromonas gingivalis (C) and Prevotella intermedia (D), as well as the cariopathogen Streptococcus mutans (E).

Conclusion. The biofilm experiments confirmed, in multispecies communities, the inhibitory activity of the Saccharomyces cerevisiae var. boulardii strain against oral pathogens, which had been demonstrated above in the agar plate experiments.

2) Effects of Strain Saccharomyces cerevisiae var. boulardii 1-3799 (boulardii yeast), Inactivated Dry Form of Strain Saccharomyces cerevisiae I-3856 (IY yeast), and their Combination

The yeast products (boulardii and IY) were each dissolved in 100 mL of PBS at 37° C. and vortexed for 3 minutes. 10 mL of these solutions was centrifuged at 6010×g for 10 minutes. The supernatants were removed and the “Boulardii” yeast pellet was suspended in BHI-2 by adjusting the concentration to an average of 5·10⁸ CFU/mL. The “IY” yeast pellet was suspended in a solution containing a sample of the bioreactor mixture diluted 1:5.
The following were placed in a first well: 1 mL of the solution of the “Boulardii” yeast suspended in BHI-2 and 1 mL of the bioreactor mixture diluted 1:5 without “IY” (“Boulardii” alone condition).
The following were placed in a second well: 1 mL of the yeast solution “IY” and of the bioreactor mixture diluted 1:5 and 1 mL of BHI-2 without “boulardii” (IY alone condition).
The following were placed in a third well: 1 mL of the “Boulardii” yeast solution suspended in BHI-2 and 1 mL of the “IY” yeast solution and of the bioreactor mixture diluted 1/5 (Boulardii+IY combination condition).
The following were placed in a fourth well: 1 mL of the bioreactor mixture diluted 1:5 and 1 mL of BHI-2. This well constitutes a control well.

The 24-well plates were incubated under microaerobic conditions (6% oxygen) for 24 hours. Then, the supernatants were removed and the biofilms attached to the hydroxyapatite discs were washed with PBS. The biofilms were detached with 1500 μL of 0.05% Trypsin-EDTA for 45 minutes at 37° C. and 230 rpm, transferred to Eppendorf tubes and centrifuged (6010×g, for 5 minutes). After removal of the trypsin, the biofilm pellet was re-suspended in 500 μL of PBS, and DNA extraction was performed according to Qiagen instructions and analyzed by qPCR.

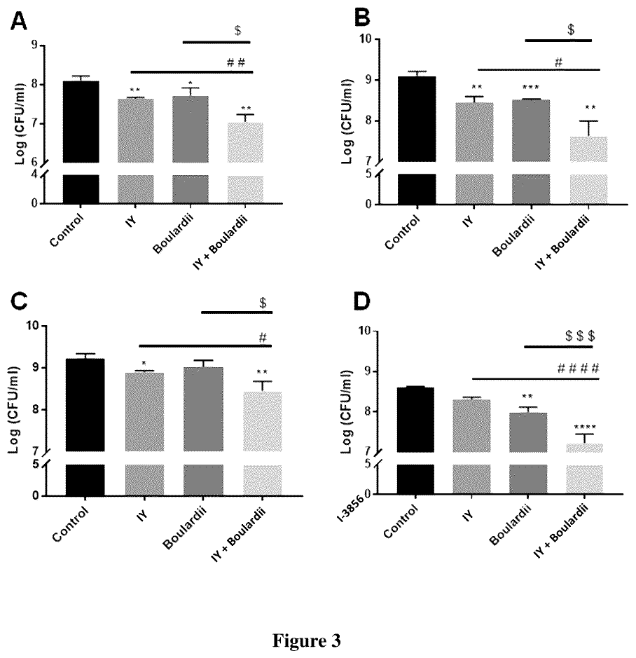

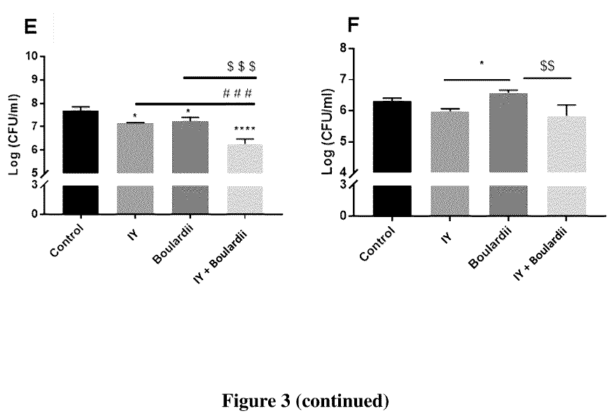

Results. The results obtained are presented in FIG. 3 and in Table 2.

TABLE 2
Difference between control values and pathogenic species
values of the biofilm (hereafter δ) in the presence of
Saccharomyces cerevisiae var. boulardii strain I-3799
(Boulardii), the inactivated dry form of Saccharomyces cerevisiae
strain I-3856 (IY), and their combination.

| Pathogenic | | δ | Sum δ IY + | δ IY + Boulardii |
|---|---|---|---|---|
| species | δ IY | Boulardii | δ Boulardii | combination |
| Aa | 0.46 | 0.38 | 0.84 | 1.1 |
| Fn | 0.65 | 0.59 | 1.24 | 1.47 |
| Pg | 0.32 | 0.2 | 0.52 | 0.77 |
| Pi | 0.3 | 0.6 | 0.9 | 1.38 |
| Sm | 0.51 | 0.43 | 0.94 | 1.4 |

Note:
The values presented in Table 2 are not absolute values. They are the difference between the control values and the pathogen values.

These results show that each of the two strains individually (Saccharomyces cerevisiae var. boulardii (Boulardii) and inactivated Saccharomyces cerevisiae (IY) and their combination are able to significantly inhibit the growth of two periodontal pathogens Aggregatibacter actinomycetemcomitans or Aa (A) and Fusobacterium nucleatum or Fn (B) and the cariogenic bacterium Streptococcus mutans or Sm (E). Furthermore, Table 2 shows that the δ of the IY+Boulardii combination is greater than the sum of the IY δ and Boulardii 8 taken individually.

According to FIG. 3 (C), the growth of Porphyromonas gingivalis or Pg (C) was inhibited by the inactivated Saccharomyces cerevisiae yeast (IY) as well as by the combination but not significantly by the Saccharomyces cerevisiae var. boulardii strain (Boulardii). Furthermore, a significant difference in inhibition between the inactivated yeast (IY) and the Boulardii+IY combination can be observed, thus demonstrating a synergistic action of the combination on the inhibition of the growth of Porphyromonas gingivalis (C), confirmed by Table 2.

According to FIG. 3 (D), the growth of Prevotella intermedia or Pi (D) was inhibited by the Saccharomyces cerevisiae var. boulardii strain as well as by the combination but not significantly by the inactivated yeast (IY). Furthermore, a significant difference in inhibition between the Boulardii strain and the Boulardii+IY combination can be observed, demonstrating a synergistic action of the combination on the inhibition of the growth of Prevotella intermedia (D), confirmed by Table 2.

Conclusion. Thus, it has been demonstrated that the combination of the Saccharomyces cerevisiae var. boulardii strain and the inactivated dry Saccharomyces cerevisiae strain was shown to induce growth inhibition of most pathogens in the multispecies model used, in contrast to the Saccharomyces cerevisiae var. boulardii strain and the inactivated Saccharomyces cerevisiae yeast taken individually which have a lesser spectrum of inhibition of oral pathogen growth.

Anti-Inflammatory Effects of Saccharomyces cerevisiae var. boulardii I-3799

The aim of the study presented below is to evaluate the potential anti-inflammatory effects of the yeast Saccharomyces cerevisiae var. boulardii CNCM 1-3799 on the inflammatory parameters of primary human monocytes and gingival fibroblasts.

### A. Evaluation of Anti-Inflammatory Potential in Primary Human Monocytes

Stimulation and Determination of Inflammatory Parameters in Primary Human Monocytes. Primary human monocytes were isolated and enriched from buffy coats of healthy human blood donors. The cells were seeded in 24-well plates (roughly 500 000 cells/mL in 1 mL) for ELISA experiments. The cells were incubated with LPS (10 ng/ml) for 24 hours. Yeast samples (5 doses ranging from 4.3^{E}6 CFU to 4.3^{E}8 CFU/mL, i.e.: 0.1 mg/mL; 1 mg/mL; 2.5 mg/mL; 5 mg/mL; 10 mg/mL) were added 30 minutes prior to LPS treatment directly into the cell culture. Dexamethasone was used as a positive control (2 doses, 1 μM and 10 μM). After 24 hours, the supernatants were removed, centrifuged, and the concentrations of MCP-1, IL-8, IL-6, IL-10, isoprostane, and PGE2 were assessed. Concentrations in EIAs (PGE2 and isoprostane, from Cayman) or ELISAs (MCP-1, IL-6, and IL-8, from eBioscience) were assessed according to the manufacturer's protocols. Each dose was studied 6 times using buffy coats from 3 different donors (n=2 per buffy coat, n=6 total).

Statistical Analysis. One-way ANOVA with Dunnett's multiple comparison tests at a significance level of 0.5.

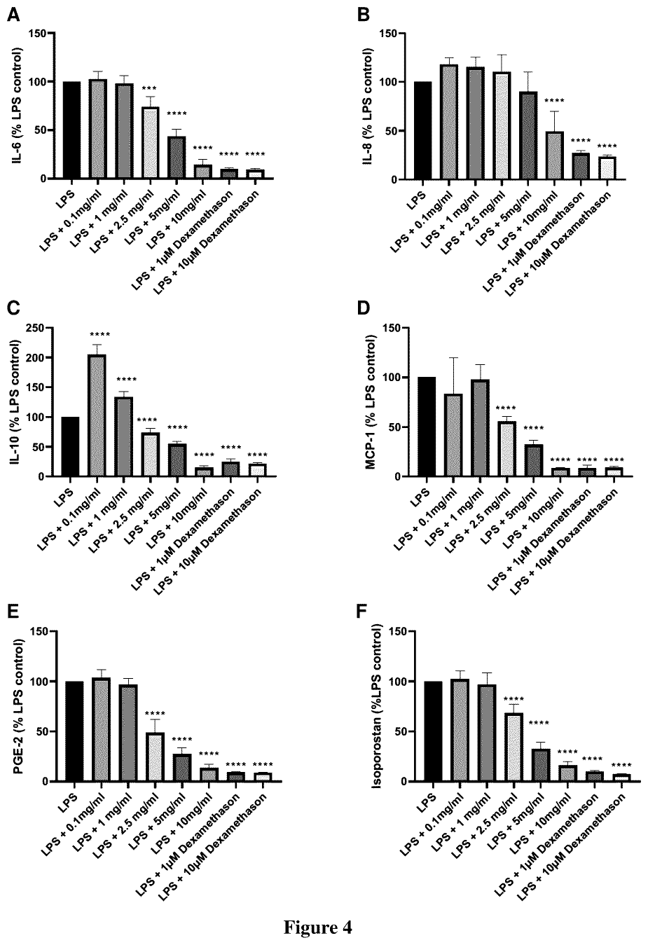

Results. The results obtained are presented in FIG. 4. These results show that the Saccharomyces cerevisiae var. boulardii CNCM I-3799 strain has a dose-dependent anti-inflammatory effect in primary human monocytes. More precisely, S. boulardii CNCM I-3799 is able to inhibit at a dose comprised between 2.5 mg/ml (1^{E}8 CFU/mL) and 10 mg/mL (4.3^{E}8 CFU/mL) the secretion of IL-6, IL-10, MCP-1, PGE-2 and isoprostane while the cytokine IL-8 is inhibited at a minimal dose of 10 mg/mL (4.3^{E}8 CFU/mL). At low doses (0.1 to 1 mg/mL or 4.3^{E}6 to 4.3^{E}7 CFU/mL), S. boulardii CNCM I-3799 is able to stimulate the secretion of the anti-inflammatory cytokine Il-10 suggesting an “immune training” effect. At higher doses, the inhibition of pro-inflammatory cytokines and PGE-2/isoprostane markers indicate an anti-inflammatory effect and an inhibition of the prostaglandin pathway involved in the onset of pain and bone destruction.

### B. Evaluation of the Anti-Inflammatory Potential on Primary Human Gingival Fibroblasts

Measurement of IL-8, IL-6, Isoprostane and PGE2 in Primary Human Gingival Fibroblasts. Cultures of primary human gingival fibroblasts supplied by Provitro (Berlin, Germany) were maintained according to the supplier's protocol. Prior to stimulation, cells were seeded in 24-well plates for ELISA experiments. The cells were incubated without (unstimulated control) or with IL-1B (10 U/mL) for 24 hours. Yeast samples were added 30 minutes before IL-1β treatment in 5 doses (0.1 mg/mL; 1 mg/mL; 2.5 mg/mL; 5 mg/mL; 10 mg/mL) (n=3). Dexamethasone was used as a positive control (1 dose, 1 μM). After 24 hours, the supernatants were collected, centrifuged, and IL-6, IL-8, isoprostane, and PGE2 concentrations were assessed by EIA (PGE2 and isoprostane, from Cayman) or ELISA (IL-6 and IL-8, from eBioscience) following the manufacturer's protocol. Each dose was studied at least 3 times.

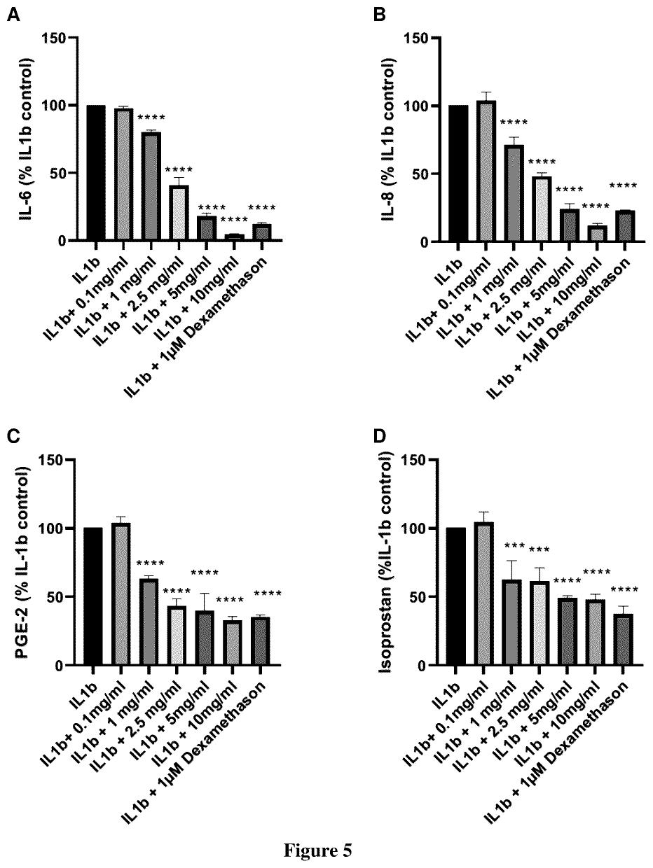

Results. The results obtained are presented in FIG. 5. These results show that the Saccharomyces cerevisiae var. boulardii CNCM I-3799 strain has a dose-dependent anti-inflammatory effect in primary human gingival fibroblasts. Specifically, S. boulardii CNCM I-3799 is able to inhibit at a dose comprised between 1 mg/mL (4.3^{E}7 CFU/mL) and 10 mg/mL (4.3^{E}8 CFU/mL) the secretion of IL-6, IL-8, PGE-2 and isoprostane. Inhibition of pro-inflammatory cytokines and PGE-2/isoprostane markers indicate an anti-inflammatory effect and inhibition of the prostaglandin pathway involved in the development of pain and bone destruction.

1. A method for treating an infectious disease of the oral cavity in a subject, the method comprising a step of administering to said subject an effective amount of:
a combination of a Saccharomyces cerevisiae var. boulardii yeast strain deposited, on Aug. 21, 2007, at the CNCM under number I-3799, with an inactivated dry form of a Saccharomyces cerevisiae yeast strain deposited, on Oct. 17, 2007, at the CNCM under number I-3856,
wherein the infectious disease of the oral cavity is dental caries, gingivitis or periodontitis, and
wherein the infectious disease of the oral cavity is a side effect of a medical treatment, or wherein the subject is a patient with immune vulnerability, or the subject exhibits reduced salivary flow, or the subject is a pregnant woman.

2. The method according to claim 1, wherein said medical treatment comprises, or consists of, administration of an immunosuppressant, phenytoin, or a calcium channel blocker to said subject.

3. The method according to claim 1, wherein said immune vulnerability is associated with Sjögren's syndrome, HIV infection, uncontrolled diabetes, endocrine disorders, malnutrition or malabsorption.

4. The method according to claim 1, wherein said subject exhibiting reduced salivary flow is an elderly subject or a smoker or a subject under stress.

5. The method according to claim 1, wherein the Saccharomyces cerevisiae var. boulardii yeast is in live dry form.

6. The method according to claim 1, wherein the combination of the Saccharomyces cerevisiae var. boulardii yeast strain with the inactivated dry form of the Saccharomyces cerevisiae yeast strain is comprised in a dietary supplement.

7. The method according to claim 6, wherein the dietary supplement is in the form of a lozenge to be sucked, a candy, a chewing gum, an orodispersible powder or a powder to be diluted in water in the form of a stick or sachet, a tablet to be sucked or chewed, a gum, a capsule, a tablet, drops, or a vial with a measuring cap.

8. The method according to claim 1, wherein the combination of the Saccharomyces cerevisiae var. boulardii yeast strain with the inactivated dry form of the Saccharomyces cerevisiae yeast strain is comprised in a parapharmaceutical or cosmetic composition.

9. The method according to claim 8, wherein the parapharmaceutical or cosmetic composition is in the form of a toothpaste, a mouthwash, an oral spray, an oral cream or gel, an orodispersible sheet, a powder to be sprinkled directly into the oral cavity, an orodispersible powder or a powder to be diluted in water in the form of a stick or sachet, or a vial with a measuring cap.

10. The method according to claim 1, wherein the combination of the Saccharomyces cerevisiae var. boulardii yeast strain with the inactivated dry form of the Saccharomyces cerevisiae yeast strain is comprised in a pharmaceutical composition, which further comprises at least one physiologically acceptable excipient.

11. The method according to claim 10, wherein the pharmaceutical composition is in a form for topical administration or for oral administration.

12. The method according to claim 10, wherein the pharmaceutical composition further comprises at least one additional pharmaceutical active principle having soothing, anti-irritant, analgesic, antalgic, anti-inflammatory, healing, antibiotic, antipyretic, or antifungal activity.

13. The method according to claim 1, wherein the combination of the Saccharomyces cerevisiae var. boulardii yeast strain with the inactivated dry form of the Saccharomyces cerevisiae yeast strain is comprised in a dental medical device.

14. The method according to claim 13, wherein said dental medical device is dental implant, a dental crown, a dental bridge, a dental onlay, or a dental prosthesis.

15. The method according to claim 1, wherein the infectious disease of the oral cavity is caused by periodontopathogenic bacteria and/or cariopathogenic bacteria.

16. The method according to claim 15, wherein the periodontopathogenic bacteria are selected from Aggregatibacter actinomycetemcomitans, Fusobacterium nucleatum, Porphyromonas gingivalis, Prevotella intermedia, and/or the cariopathogenic bacterium Streptococcus mutans.
